(19) United States
(12) Patent Application Publication
(10) Pub. No.: US 2026/0013798 A1
(43) Pub. Date: Jan. 15, 2026

(54) HEART FAILURE DECOMPENSATION APPARATUS AND METHOD

(71) Applicants: Podimetrics, Inc., Somerville, MA (US); MUSC Foundation for Research Development, Charleston, SC (US)

(72) Inventors: David R. Linders, Waltham, MA (US); Michele Esposito, Mount Pleasant, SC (US); Graydon Mears, San Antonio, TX (US); Mustafa Karabas, Concord, MA (US)

(21) Appl. No.: 19/264,275

(22) Filed: Jul. 9, 2025

Related U.S. Application Data
(60) Provisional application No. 63/668,884, filed on Jul. 9, 2024.

(51) Int. Cl. A61B 5/00; A61B 5/0205; G01G 19/50; G16H 10/60; G16H 50/20; G16H 50/30

(52) U.S. Cl. CPC A61B 5/7275; A61B 5/02055; A61B 5/7475; G01G 19/50; G16H 10/60; G16H 50/20; G16H 50/30

(57) ABSTRACT

A method and system for detecting risk of heart failure decompensation of a patient receives both weight information of the patient and foot temperature information of the patient. Using that received information, the method/system determines whether a change in weight and foot temperature indicates a pattern indicative of heart failure decompensation. Next, the method produces output information indicating a result of that determination.

### PRIORITY

This patent application claims priority from provisional U.S. patent application No. 63/668,884, filed Jul. 9, 2024, entitled, “HEART FAILURE DETECTION APPARATUS AND METHOD,” and naming David R Linders, Michele Esposito, Graydon Mears, and Mustafa Karabas as inventors, the disclosure of which is incorporated herein, in its entirety, by reference.

### FIELD

Illustrative embodiments of the invention generally relate to a medical device and, more particularly, various embodiments of the invention relate to managing heart failure.

### BACKGROUND

Heart failure is a condition in which the heart cannot pump blood effectively enough to meet the body's needs. This can occur because the heart muscle is either too weak to contract with sufficient force or too stiff to fill properly with blood. When the heart is weak, it struggles to push blood out to the body. When it is stiff, it cannot fill adequately between beats. Both issues can lead to a buildup of fluid in the body, resulting in symptoms such as shortness of breath, fatigue, weight gain, and swelling in the legs and feet.

Managing heart failure requires a multifaceted approach to improve heart function and alleviate symptoms. Key strategies include lifestyle changes such as following a heart-healthy diet, engaging in regular physical activity, and avoiding smoking and excessive alcohol consumption. Medications play a vital role by reducing fluid buildup, lowering blood pressure, and easing the heart's workload. In some cases, medical devices like pacemakers or surgical interventions may be necessary to support heart function. Ongoing care through regular check-ups is essential to monitor the condition and adjust treatment plans as needed.

Heart failure decompensation refers to a sudden worsening of heart failure symptoms. This can be triggered by factors such as infections, poor adherence to medications or dietary guidelines, or the emergence of new cardiac issues. Decompensation often necessitates urgent medical intervention—typically hospitalization—to stabilize the patient. Treatment may involve oxygen therapy, oral or intravenous medications, and addressing the underlying cause of the exacerbation. Preventing decompensation requires diligent management of heart failure and prompt attention to any new health concerns. Early detection of heart failure decompensation is critical for preserving quality of life and minimizing the risk of hospitalization.

### SUMMARY OF VARIOUS EMBODIMENTS

In accordance with one embodiment of the invention, a method of identifying a risk of heart failure decompensation of a patient receives first weight information and earlier weight information of the patient, as well as first foot temperature information and earlier foot temperature information of patient. The first weight information is temporally spaced (i.e., represents different times) from the earlier weight information. The first foot temperature information and earlier foot temperature information of the patient each are associated with at least one portion of the patient's foot and, like the weight information, also are temporally spaced from the earlier foot temperature information.

At least one of the first weight information and the first foot temperature information are produced by and communicated from a platform having at least one weight sensor to detect weight and at least one temperature sensor configured to determine the temperature at different spaced apart portions of the foot. The platform may be one of an open platform and a closed platform. The method uses a multi-modal model executing on at least one computing device and configured to analyze physiological data to determine whether:
1) the first weight information,
2) the earlier weight information,
3) the first foot temperature information, and
4) the earlier foot temperature information,
collectively shows a physiological pattern indicative of heart failure decompensation. Finally, the method produces output information for use in clinical decision-making or patient monitoring relating to whether the model determined a pattern indicative of heart failure decompensation.

The method may use the model by determining whether the first weight information and earlier weight information indicate a change of weight, and determining whether the first foot temperature information and the earlier foot temperature information indicate a change in foot temperature. As such, the model uses those determinations to determine whether 1-4 collectively indicates heart failure decompensation. Moreover, the first foot temperature information may be associated with a given two or more portions of the patient's foot, while the earlier foot temperature information also are associated with the (same) given two or more portions of the patient's foot. To provide information to a user, a display device output may produce indicia relating to whether the model determined a pattern indicative of heart failure decompensation.

Among other things, the earlier weight information may include a baseline of weight information, while the earlier foot temperature information may include a baseline of temperature information. Different temperature types may be used. For example, the temperature information may be a representation of the temperature of the whole foot as a median or average temperature across measurements before the first temperature measurement.

Some embodiments also use fluid accumulation information relating to the patient. In that case, the model may also use fluid accumulation information to determine whether a pattern indicative of heart failure decompensation exists. This information may be obtained by using bioimpedance sensors to determine fluid accumulation in the patient.

Open and closed platforms may be used. For example, the method may receive the first weight information from an open platform having weight sensors. In a similar manner, the method may receive foot temperature information from an open platform having one or more temperature sensors.

Preferred embodiments of the model may include one or more of a sensitivity adjusted model, a statistical model, a mathematical model, and a machine learning technique. To simplify the platform, the model may be executed remote from its open or closed platform. In addition to objective information received from sensors, some embodiments receive answers from the patient relating to a plurality of questions. The model uses at least one of the answers and items 1-4 to determine whether a pattern indicative of heart failure decompensation exists.

In accordance with another embodiment, a method of identifying a risk of heart failure decompensation of a patient receives first fluid accumulation information and earlier fluid accumulation information of the patient. The first fluid accumulation information is temporally spaced from the earlier fluid accumulation information. The method also receives first foot temperature information and earlier foot temperature information of the patient. Each set of temperature information is associated with at least one portion of the patient's foot and is temporally spaced from the other.

At least one of the first fluid accumulation information and the first foot temperature information is produced by and communicated from a platform that includes at least one fluid accumulation sensor to detect fluid accumulation and at least one temperature sensor configured to determine temperature at different spaced apart portions of the foot. The platform may be either an open platform or a closed platform.

This method uses a multi-modal model executing on at least one computing device and configured to analyze physiological data to determine whether:
1. the first fluid accumulation information,
2. the earlier fluid accumulation information,
3. the first foot temperature information, and
4. the earlier foot temperature information,
collectively show a physiological pattern indicative of heart failure decompensation. The method then produces output information for use in clinical decision-making or patient monitoring based on whether the model determined such a pattern.

In accordance with other embodiments, a heart failure management system includes an input configured to receive multi-modal physiological information from a platform. The platform includes at least one temperature sensor configured to produce foot temperature information associated with at least one portion of the patient's foot, and at least one weight sensor configured to produce patient weight information. The platform includes at least one of an open platform and a closed platform.

The system also includes an analysis engine with a multi-modal model configured to analyze physiological information received at the input. The analysis engine is configured to receive first foot temperature information produced by the temperature sensor and earlier foot temperature information of the patient. The first foot temperature information is temporally spaced from the earlier foot temperature information, and both are associated with at least one portion of the patient's foot. The analysis engine is further configured to receive first weight information and earlier weight information of the patient, with the first weight information temporally spaced from the earlier weight information. The multi-modal model of the analysis engine is configured to determine whether:
1. the first weight information,
2. the earlier weight information,
3. the first foot temperature information, and
4. the earlier foot temperature information,
collectively show a pattern indicative of heart failure decompensation.

The system includes an output configured to produce output information for use in clinical decision-making or patient monitoring relating to whether the multi-modal model determined a pattern indicative of heart failure decompensation.

In accordance with yet other embodiments, a heart failure management system includes an input configured to receive multi-modal physiological information from a platform. The platform includes at least one temperature sensor configured to produce foot temperature information associated with the temperatures of at least one portion of the patient's foot. The platform also includes at least one fluid sensor configured to produce patient fluid accumulation information. The platform includes at least one of an open platform and a closed platform.

The system includes an analysis engine with a multi-modal model configured to analyze physiological information received at the input. The analysis engine is configured to receive first foot temperature information produced by the temperature sensor and earlier foot temperature information of the patient. The first foot temperature information is temporally spaced from the earlier foot temperature information, and both are associated with one or more portions of the patient's foot. The analysis engine is further configured to receive first fluid accumulation information and earlier fluid accumulation information of the patient, with the first fluid accumulation information temporally spaced from the earlier fluid accumulation information. The multi-modal model of the analysis engine is configured to determine whether:
1. the first fluid accumulation information,
2. the earlier fluid accumulation information,
3. the first foot temperature information, and
4. the earlier foot temperature information,
collectively show a pattern indicative of heart failure decompensation.

The system also includes an output configured to produce output information for use in clinical decision-making or patient monitoring relating to whether the multi-modal model determined a pattern indicative of heart failure decompensation.

Illustrative embodiments of the invention are implemented as a computer program product having a computer usable medium with computer readable program code thereon. The computer readable code may be read and utilized by a computer system in accordance with conventional processes.

### BRIEF DESCRIPTION OF THE DRAWINGS

Those skilled in the art should more fully appreciate advantages of various embodiments of the invention from the following “Description of Illustrative Embodiments,” discussed with reference to the drawings summarized immediately below.

FIG. 1 schematically shows an open platform that may be configured in accordance with illustrative embodiments of the invention.

FIG. 2 schematically shows an exploded view of one type of open platform that may be configured in accordance with illustrative embodiments of the invention.

FIGS. 3A and 3B respectively schematically show non-contact temperature sensors and contact temperature sensors on an open platform in accordance with illustrative embodiments.

FIG. 4A schematically shows an array of temperature sensors in accordance with illustrative embodiments.

FIG. 4B schematically shows bioimpedance measurements through feet and hands in accordance with illustrative embodiments.

FIG. 5 schematically shows a network implementing of illustrative embodiments of the invention.

FIG. 6A schematically shows a measurement system and related systems in illustrative embodiments.

FIG. 6B schematically shows another measurement system and related systems in illustrative embodiments.

FIG. 7 shows a process of collecting and analyzing sensor data for foot and weight to predict heart failure decompensation risk in accordance with one embodiment.

FIG. 8 shows a process of collecting and analyzing weight, foot temperature, and body fluid composition to predict risk of heart failure decompensation risk in accordance with illustrative embodiments.

FIG. 9 graphically shows experimental data from illustrative embodiments.

### DESCRIPTION OF ILLUSTRATIVE EMBODIMENTS

Illustrative embodiments use multiple types of physiological data to more accurately predict and support timely treatment of decompensating heart failure. To that end, illustrative systems use temporally spaced measurements of both patient weight and foot temperature to identify patterns indicative of heart failure decompensation. These measurements may be collected using an open or closed platform equipped with weight and/or temperature sensors. A multi-modal model analyzes the combined data to determine whether a physiological pattern is present, and produces output information for use in clinical decision-making or patient monitoring. Details of illustrative embodiments are discussed below.

Heart failure (HF) affects more than six million individuals nationwide and is a leading cause of hospitalizations for individuals over the age of 65. Despite significant advancements in guideline-directed medical and device therapy, readmission rates for this cohort remain high. In some studies, approximately one-quarter of HF patients are readmitted within 30 days, while half are readmitted within 6 months.

One way of monitoring both the presence and severity of HF-related decompensation preceding hospitalization is an invasive procedure called “right heart catheterization” (RHC). RHC provides direct measurement of right heart filling pressures and allows for the calculation of cardiac output and cardiac index (cardiac output normalized to body surface area). Notably, previous research has found a correlation between cardiac output measurements from RHC and great/big toe skin temperature. In one study, authors discovered that big toe temperature, in contrast to temperatures measured in the distal third finger, deltoid, lateral thigh, and rectum, was a sensitive indicator of cardiac output. The investigators also found that big toe temperature accurately predicted patient outcomes 67% of the time, with lower temperatures indicating lower survival prospects.

Daily body weights, as a nonspecific metric of fluid accumulation, have been shown to increase prior to a HF hospitalization.

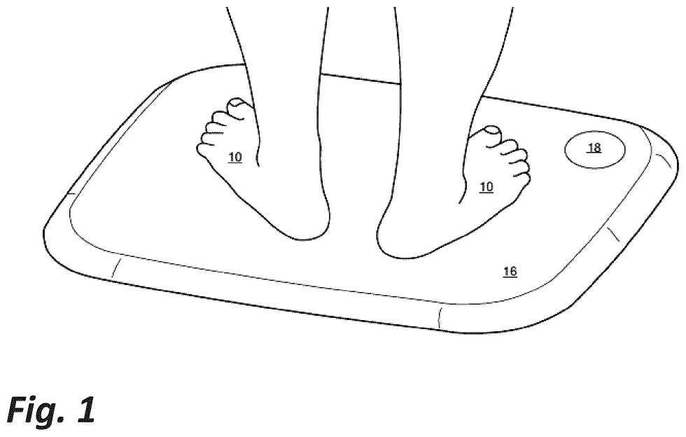

To more accurately predict and treat HF, illustrative embodiments use a platform that can measure foot temperature and body weight. Using the data received from sensors on the platform, illustrative embodiments can manage HF. Various implementations may use an open platform or a closed platform. FIG. 1 schematically shows an open platform implementation configured in accordance with illustrative embodiments.

FIG. 1 schematically shows one form factor, in which a patient/user is standing on an open platform 16 that gathers data about that user's feet 10. In this example, the open platform 16 is in the form of a floor mat or slightly raised apparatus (like a bathroom scale) placed in a location where the patient regularly stands, such as in front of a bathroom sink, next to a bed, in front of a shower, on a footrest, or integrated into a mattress. As an open platform 16, the patient simply may step on the top sensing surface of the platform 16 to automatically initiate the monitoring process. Accordingly, this and other form factors favorably do not necessarily require that the patient affirmatively decide to interact with the platform 16. Instead, many expected form factors are configured to be used in areas where the patient frequently stands during their day without a foot covering. Alternatively, however, some embodiments require the user to select an “on” or “start” button or the like (e.g., on the platform 16, in an application on the user's phone, or other location) to initiate the process.

The noted bathroom mat, bathroom scale, or rug form factors are but several of a wide variety of different potential form factors. Others may include a platform 16 resembling a stand, a footrest, a console, a tile built into the floor, or a more portable mechanism that receives at least one of the feet 10. The implementation shown in FIG. 1 has a top surface area that is larger than the surface area of one or both feet 10 of the patient.

The open platform 16 also has indicia or display 18 on its top surface that can have any of a number of functions. For example, the indicia can turn a distinct color or sound an alarm after the readings are complete, show the progression of the process, or display results of the process. Of course, the indicia or display 18 can be at any location other than on the top surface of the open platform 16, such as on the side, or a separate component that communicates with the open platform 16. In fact, in addition to, or instead of, using visual or audible indicia, the platform 16 may have other types of indicia, such as tactile indicia/feedback, our thermal indicia.

To monitor the health of the patient's foot 10 (discussed in greater detail below), the platform 16 of FIG. 1 gathers weight data, as well as temperature data from a plurality of different locations on the sole of the foot 10. Specifically, low blood flow typically produces temperature changes/drops in the extremities, while fluid accumulation/retention increases a patient's weight. This data provides relevant information used to manage HF.

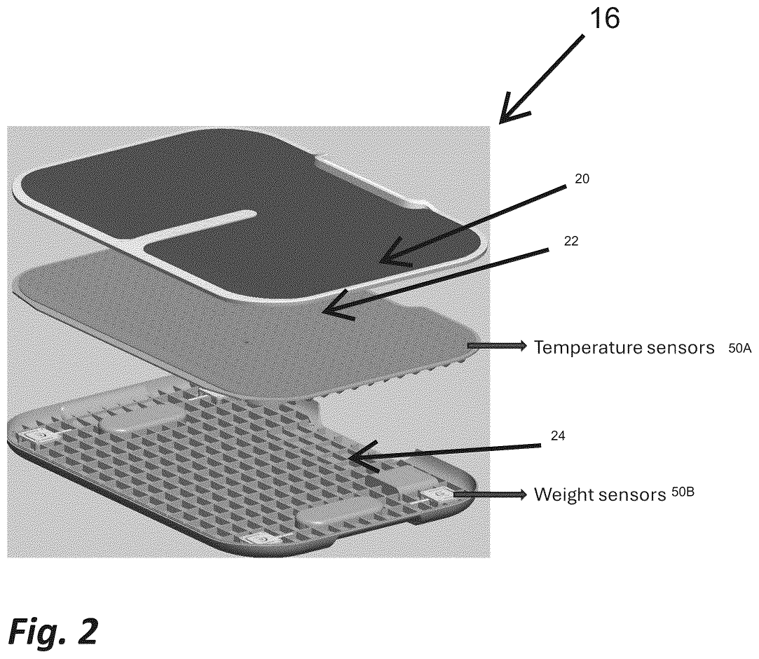

FIG. 2 schematically shows an exploded view of the open platform 16 configured and arranged in accordance with one embodiment of the invention. Of course, this embodiment is but one of a number of potential implementation and, like other features, is discussed by example only. As shown, the platform 16 is formed as a vertically stacked assembly of functional layers, including a top cover 20 to directly contact the user, one or more sensor layers, a printed circuit board (PCB 22, which may be part of the noted sensor layers), a structural base 24, and a non-skid bottom surface. The overall geometry of the platform 16 is generally rectangular or square, with a surface area sufficient to accommodate both feet of a standing user. The platform 16 has a low-profile design—typically less than 2 inches in height—to reduce tripping risk and facilitate daily use in home or clinical environments.

The top cover 20 layer may be formed from a thermally conductive yet durable material, such as a thin polymer or composite sheet, which facilitates efficient conductive heat transfer from the foot 10 to the underlying temperature sensors 50A. This layer may also include visual or tactile indicia to guide foot placement and may be textured or coated to enhance comfort and slip resistance.

Beneath the cover is a temperature sensor layer, which includes the rigid, flexible, or semiflexible PCB 22, having an array of temperature sensors 50A. These temperature sensors 50A may include one or more of thermistors, thermocouples, resistive temperature detectors (RTDs), one or more thermochromic sensors, and/or infrared sensors, and are arranged in a two-dimensional grid to capture temperature data from multiple spaced-apart anatomical regions of the foot 10, such as the heel, arch, metatarsal heads, and toes. The PCB 22 may be fabricated from a flexible polyimide substrate, flex circuit material, or a rigid FR-4 material, depending on the desired mechanical properties. In some embodiments, the PCB 22 is segmented or contoured to conform to the natural curvature of the foot 10. For example, the top surface may be flexible to contour to the foot 10.

A weight sensor layer is positioned beneath (e.g., in the base 24) or integrated with the temperature sensor layer. This layer may include one or more load cells, strain gauges, or pressure-sensitive resistive or capacitive elements that measure the user's body weight when standing on the platform 16. The weight sensors 50B may be distributed across the platform 16 to capture both total weight and weight distribution, which can be useful for balance assessment or gait analysis. In some embodiments, the weight sensors 50B are mounted on a subframe or embedded within a compressible support layer to isolate them from lateral forces.

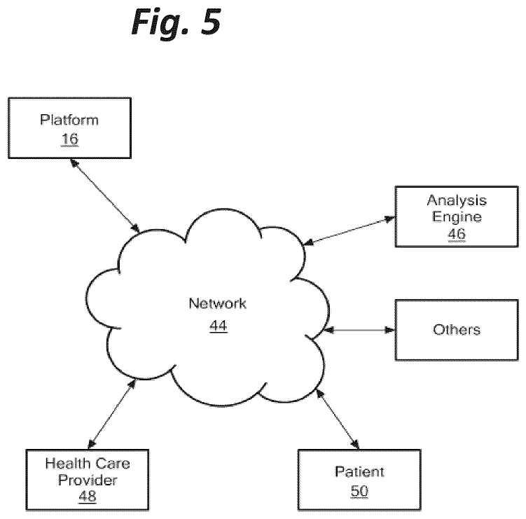

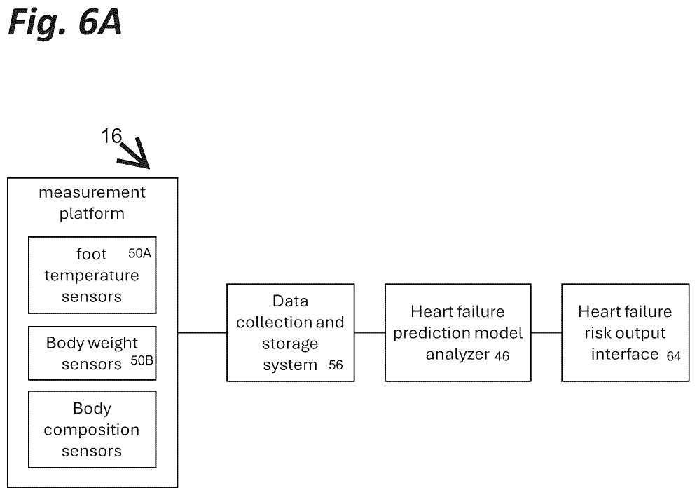

The rigid structural base 24 provides structural support and houses the electronics, including a microcontroller, power supply, wireless communication module, and data storage (shown schematically in subsequent FIG. 6A). The base 24 may be formed from injection-molded plastic, aluminum, or composite materials, and may include internal compartments or mounting brackets for the PCB 22 and sensor assemblies. The base 24 also serves as a protective enclosure for the electronics and may include thermal shielding or vibration damping features.

The bottom surface of the rigid base 24, which makes up the bottom surface of the platform itself, includes a non-skid layer, such as a rubberized mat or textured polymer sheet, to prevent slipping during use. This layer may also include leveling feet or compliance zones to ensure stable contact with the floor.

The integration of temperature and weight sensors 50A and 50B into a single platform 16 allows for simultaneous or sequential acquisition of physiological data with minimal user effort. The platform 16 may be configured for use in a fixed location (e.g., beside a bed or sink) or as a portable device. In some embodiments, the platform 16 includes a user-facing display 18 or indicator lights to provide real-time feedback, such as measurement status or alerts. Other embodiments may transmit data wirelessly to a remote processor or mobile device for further analysis.

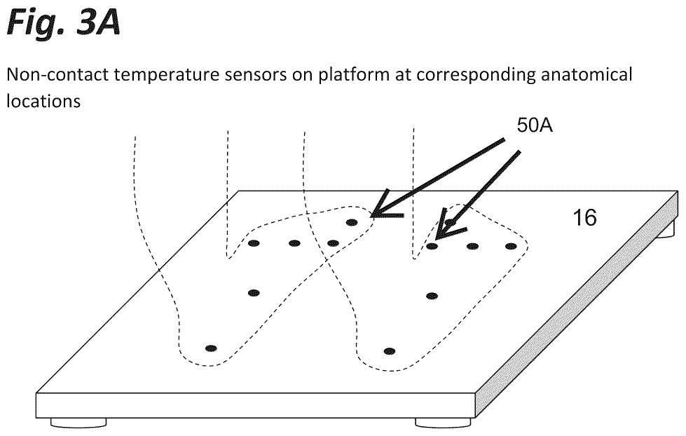

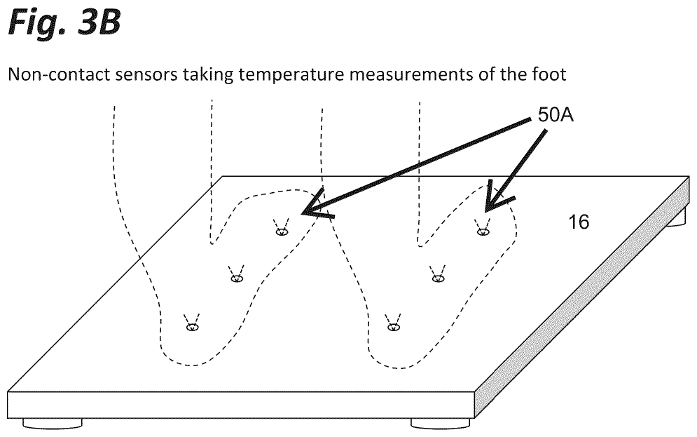

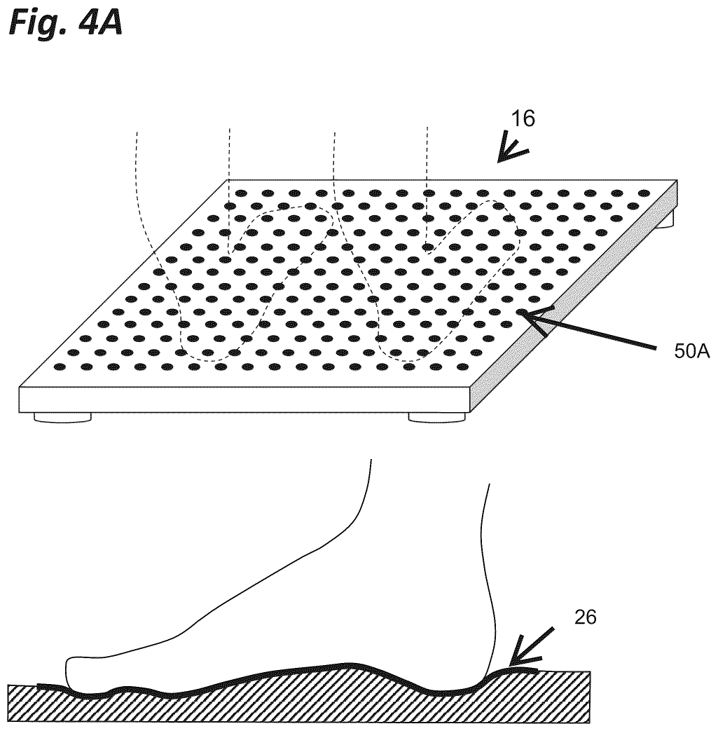

Some embodiments make separate weight and temperature measurements with different devices. For example, body weight can be measured using a standard weight scale, while fluid composition may be determined through bioimpedance sensors (FIG. 4B, discussed below), which can be attached to the platform 16 (FIG. 3B) or part of a handheld device (FIG. 4B). Foot temperature also can be measured using a separate device, such as a thermal and/or standard camera, a handheld dermal thermometer, or a probe that is affixed to the foot 10. The use of separate devices for each measurement allows users to focus on specific health metrics one at a time, using tools that are specialized for each function. Whether it is the precision of a dedicated foot temperature probe or the familiarity of a handheld bioimpedance sensor, these separate devices can cater to the varied preferences of users.

In some embodiments, the open platform 16 includes a number of other functional components, such as a microcontroller, battery, memory, and wireless communication module (e.g., Bluetooth or Wi-Fi) to locally process and transmit data to a remote server or mobile device.

Rather than using an open platform 16, alternative embodiments may implement a closed platform (also referred to herein using reference number “16”), such as a shoe, insole, or sock, which can be worn by the patient either continuously or on an as-needed basis. These closed platforms 16 are configured to maintain direct and consistent contact with the foot 10, enabling the collection of physiological data during normal daily activities without requiring the patient to stand on a separate device.

For example, the insole of a patient's shoe or boot may be embedded with one or more temperature sensors 50A and pressure or weight sensors 50B, along with communication interface or connections for a communication interface. The temperature sensors 50A may be positioned at key anatomical locations—such as the heel, arch, metatarsal heads, and hallux—to capture localized or averaged foot temperature data. These sensors 50A may be thermistors, thermocouples, or resistive temperature devices (RTDs), and may be mounted on a flexible printed circuit board (PCB 22) that conforms to the shape of the insole.

As with the open platform 16 example above, the weight or pressure sensors 50B may include piezoelectric, capacitive, or resistive elements capable of detecting total body weight, weight distribution, or changes in pressure over time. These sensors may be embedded within the insole's mid-layer or integrated into a multilayer laminate structure that includes cushioning and support materials.

The outer layers of the closed platform 16 may be formed from breathable, moisture-wicking textiles or elastomeric materials to ensure comfort and durability. In some embodiments, the closed platform 16 includes a microcontroller, battery, and wireless communication module (e.g., Bluetooth or Wi-Fi) to locally process and transmit data to a remote server or mobile device. Other embodiments may store data locally for later retrieval.

Closed platforms 16 may also include alignment features or anatomical contours to ensure consistent sensor placement and reliable data acquisition. These platforms are particularly useful for ambulatory monitoring, remote patient management, or integration into rehabilitation programs where continuous or passive data collection is desirable.

To accurately monitor foot temperature, various sensor configurations on different modalities (e.g., open platforms or close platforms 16) can be employed. One approach involves using one or more rigid sensors comprising thermistors or other contact or non-contact sensors to take temperature measurements over the sole of the foot 10. This could involve a two-dimensional array of thermal sensors 50A (e.g., a plurality of contact thermochromic sensors arranged on a grid, such as that shown in FIG. 4A) to measure the temperature across the entire surface of the soles. Such an array allows for comprehensive temperature mapping, providing detailed information about different areas of the foot 10. Alternatively, a single temperature sensor 50A (e.g., a single thermochromic member, layer, or film) could be used to measure the temperature at one specific location.

Another option uses asymmetrically positioned thermal sensors 50A throughout (e.g., in a non-grid patter), or a pattern of multiple thermal sensors 50A that measure temperatures at several strategic locations on the foot 10. For example, FIG. 3A schematically shows temperature sensors 50A at the heel and big toe, among other places. This provides a balance between detail and simplicity. Other embodiments may use one or more thermochromic sensor(s) and/or thermal or non-thermal cameras. The camera(s) can work with the thermochromic sensors to determine foot temperature. To ensure accurate placement, a physical feature might be necessary to guide the foot 10 into the correct position so that the sensor 50A aligns properly with the targeted measurement area.

In contrast to sensors 50A on the rigid surface as above, some embodiments may use a flexible sensor layer/array 26 (e.g., see FIG. 4A, bottom image) with temperature sensors 50A. While this setup could also involve an array of sensors 50A arranged on a grid to cover the entire sole of the foot 10, the flexible nature of the array allows it to conform closely to the foot's contours (e.g., the arch), potentially providing more accurate and comfortable measurements. For example, as noted above, see FIG. 4A with an array of sensors 50A on a flexible surface or rigid surface. Alternatively, a single or smaller number of sensors 50A could be mounted on a flexible surface (e.g., on an open platform 16 or a closed platform 16), which would then make close contact with the bottom surface of the foot 10. This flexibility ensures good contact and consistent measurements, even as the foot 10 moves or shifts slightly. Both rigid and flexible sensor configurations have their respective advantages and can be chosen based on specific needs and preferences in the application of foot temperature monitoring.

In addition to thermistors and thermochromic sensors, other types of contact temperature sensors 50A can be employed for monitoring foot temperature. These include thermocouples and resistive temperature devices (RTDs), which the foot 10 contacts when standing on the platform 16. Thermocouples, known for their rapid response and wide temperature range, can provide accurate readings quickly. RTDs, on the other hand, offer high precision and stability over time. Both sensor types can be integrated into a system that ensures reliable contact with the foot 10, enabling precise temperature measurement.

As shown in FIG. 3B, non-contact temperature sensors 50A present another approach to foot temperature monitoring. These sensors 50A can be installed on the platform 16 near the foot 10 and include optical sensors such as near-infrared sensors. When the foot 10 is placed on the surface, these sensors 50A can measure temperature without direct contact. This method minimizes discomfort and reduces the risk of sensor displacement, ensuring consistent and accurate readings. The use of optical sensors allows for quick and hygienic temperature assessments, which can be particularly useful in settings where multiple users are involved.

As suggested above, thermal cameras offer another alternative for foot temperature measurement. One configuration involves placing the thermal camera on a support mechanism (e.g., a tripod) with a transparent window underneath. This window could either have apertures to allow the thermal camera to view through the surface or be made from a material transparent to infrared light. The thermal camera can detect the temperature change of the platform 16 as the foot 10 heats it up over time, providing a detailed thermal image of the foot's temperature distribution. This could also be used in conjunction with other temperature sensors 50A.

Alternatively, an apparatus could hold the feet in a specific position relative to the thermal camera. For instance, a thermal camera could be mounted on the end of a long-handled, extendable stick, like a “selfie stick.” This stick could include features to position the feet at the correct angle and distance to the lens, ensuring precise measurements. Another design could involve an apparatus that holds the heels of the feet in position while keeping the camera at a fixed distance and angle, placed on the floor for stability.

A more portable solution could involve a thermal camera on a handheld device, allowing the user to image the foot 10 while holding the device. This method provides flexibility and convenience, enabling users to take temperature measurements in various settings and positions.

Infrared (IR) sensors provide a versatile option for foot temperature measurement due to their non-contact nature and quick response time. One approach involves using an infrared (single point) sensor positioned at the end of a handheld probe. This configuration allows manual measurement of foot temperature, providing flexibility and ease of use. The user can direct the probe to specific areas of the foot 10 to obtain targeted temperature readings.

Alternatively, infrared sensors can be integrated into an open platform 16 to measure foot temperature at one or more fixed points when the foot 10 is placed upon the platform 16. This setup allows for consistent and repeatable measurements, as the sensors 50A are positioned to capture temperature data from predetermined locations. Multiple sensors 50A can be used in either a stationary or automated setup.

The temperature data collected from these sensors 50A can be managed in different ways. One method involves manual data collection by the user, who records the temperature readings for subsequent analysis. This approach is straightforward and allows the user to have direct control over the data recording process.

Alternatively, temperature data can be collected automatically when the user takes a scan or measurement. With this automated approach, the data is saved (e.g., in local or in remote memory), ensuring that all readings are accurately captured without requiring manual input. Furthermore, the data can be processed locally, and/or transmitted to a remote server for analysis (e.g., some data could be processed locally, some could be processed remotely, or all could be processed remotely). This allows for the combination of temperature data with other information from the apparatus, providing a comprehensive dataset for monitoring and analysis. Automated data collection and remote transmission enhance the efficiency and accuracy of temperature monitoring, making it easier to track changes over time and identify potential issues promptly.

Like the temperature sensors 50A, the weight sensors 50B can be positioned and used in any of a variety of manners. Indeed, the positions in FIG. 2 are just illustrative. Specifically, weight measurement can be effectively conducted using the open platform 16 equipped with one or more of distinct types of weight sensors 50B. These sensors 50B are activated when and/or after the user stands on the platform 16, allowing for accurate weight assessment through different mechanisms.

One approach involves using strain gauge technology or load cells to measure the force applied to the platform 16 by the user's weight. Load cells convert the applied force into an electrical signal, which is then processed to determine the user's weight with high accuracy and repeatability. This method is beneficial for its precision and reliability.

Another method employs spring-loaded mechanisms to calculate the force applied by the user's weight. In this setup, the displacement of the spring under the user's weight is measured in several ways to determine the applied force. This displacement is then translated into a weight measurement. This method leverages the mechanical properties of springs and can be an effective, low-cost solution for weight measurement.

A more advanced approach involves using a pressure distribution plate, which measures the pressures over the entire surface of the feet. Individual pressures are measured on a grid or a pattern that substantially covers both feet. The sum of these pressures and the area over which they are applied can be used to calculate the total force, corresponding to the user's weight. Pressure sensors for this purpose can be fabricated using various technologies, including resistive, capacitive, or optical methods. This approach not only measures weight but also provides valuable data on pressure distribution, which can be useful for assessing foot health and gait analysis.

The user's weight and/or other visual indicia can be displayed on a screen or other display 18 integrated with the platform 16, allowing the user to easily read and record their weight. For more advanced setups, the device can automatically collect the weight data and store it internally. This data can then be transmitted to a remote server for further analysis, enabling long-term monitoring and integration with other health data. Automatic data collection and remote transmission enhance the utility of the weight measurement system, providing users and healthcare providers 48 with detailed insights into weight trends and related health metrics.

Body fluid composition analysis also can be helpful for monitoring overall health, particularly for heart failure. This can be effectively integrated into the platform 16 and achieved using Bioimpedance Spectroscopy (BIS) or bioimpedance analysis (BIA), which are non-invasive methods that estimate body composition from electrical signals passed through the body.

Bioimpedance analysis involves passing an alternating current (AC) electrical signal through the body's intracellular and extracellular water at different frequencies (e.g., see FIG. 4B, which shows a bioimpedance through the hands and feet). This technique is well-regarded for its reliability in estimating body composition. By applying small currents at one or more frequencies to specific parts of the body (such as one foot 10), the composition of various compartments (including two legs, two arms, and the trunk) can be estimated. These estimates cover metrics such as Fat Mass (FM), Fat-Free Mass (FFM), Bone Mass, Total Body Water, intracellular fluids, extracellular fluids, and other derivative metrics.

Impedance measurements at specific frequencies enable the estimation of Total Body Water (TBW), which is related to the length of the electrical conduction path and generally proportional to the user's height. From TBW, FFM can be calculated using the formula TBW=0.73*FFM for a normally hydrated body. The user's Body Weight (BW) is also a crucial input for these calculations, as FM can be derived from FFM using the formula FM=BW−FFM.

Bioimpedance analysis can be performed at a single frequency, typically around 50 kHz, known as Single-Frequency Bioimpedance Analysis (SF-BIA). Alternatively, it can be conducted over a range of frequencies (BIS). BIS improves the accuracy of body composition calculations by accounting for the varying conductivity of different tissue types, offering a more comprehensive analysis.

Extracellular water content, measured by low-frequency impedance, is a known marker of fluid accumulation associated with decompensated heart failure. Specifically, during heart failure decompensation, the heart's insufficient pumping action leads to the reabsorption of water in the kidneys and the expansion of extracellular fluid. This retained fluid, especially around the lungs, can cause shortness of breath. Changes in extracellular fluid, as measured by segmental or whole-body bioimpedance, serve as early indicators of heart failure decompensation, potentially preceding symptom onset.

On-platform foot-to-foot segmental measurement involves placing electrodes on the platform 16, allowing the current to flow from one electrode through one foot 10, up the leg, down the other leg, and out through the other foot 10 to the second electrode. This method primarily measures the lower limbs' body composition. Peripheral edema, associated with HF, is significantly correlated with lower extracellular resistance in foot-to-foot segmental analysis.

In contrast, handheld devices can perform hand-to-hand segmental measurements. The user holds a controller unit 28 with electrodes, and the current flows from one electrode through one hand, up the arm, across the chest, down the other arm, and out through the second electrode. This method primarily measures the upper body's composition. Pulmonary congestion, another heart failure symptom, is significantly correlated with lower extracellular resistance in hand-to-hand segmental measurements.

An alternative system includes electrodes for both lower limb and upper body composition measurements, which can be combined or compared to enhance body composition analysis. A preferred embodiment compares these measurements to isolate the water content in the upper body. By measuring foot-to-foot and hand-to-hand impedance, it becomes possible to differentiate between peripheral edema and pulmonary congestion more accurately, thus improving monitoring or diagnosis.

Tracking body fluid in the arms and trunk over time can reveal deviations from the patient's baseline, indicating additional fluid retention resulting from heart failure decompensation. This comprehensive approach provides a robust tool for managing heart failure and other conditions associated with fluid imbalance.

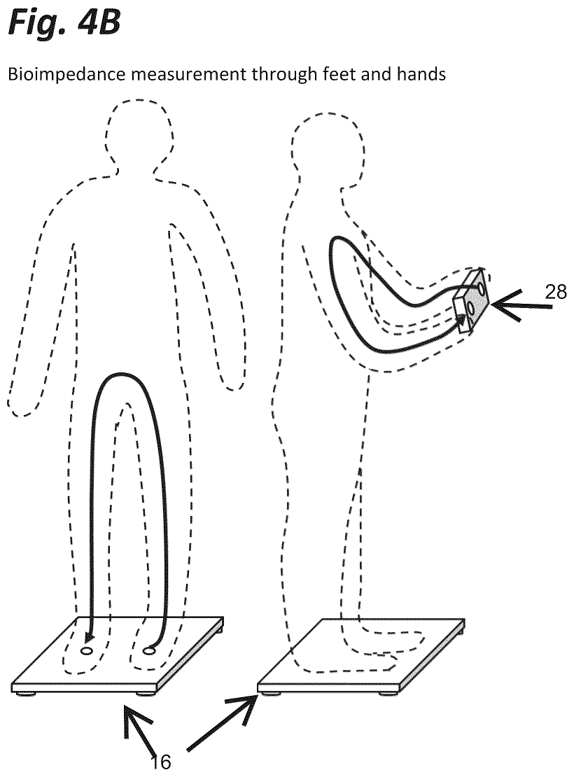

Illustrative embodiments may locate logic for monitoring and analyzing heart failure health at another location. For example, such logic may reside on a remote computing/server device. To that and other ends, FIG. 5 schematically shows one way in which a thermal camera, open platform 16, closed platform 16, or other modality (shown schematically in FIG. 5 as “Platform 16” but applicable to other modalities) can communicate with a larger data network 44 in accordance with various embodiments of the invention.

As shown, the platform 16 may connect with the Internet through a local router, through its local area network, or directly without an intervening device. This larger data network (e.g., the Internet) can include any number of different endpoints that are also interconnected. For example, the platform 16 may communicate with an analysis engine 46 (discussed below) that uses a model to analyze thermal, weight, or other physiological data it receives from the platform 16. The platform 16 also may communicate with a healthcare provider 48, such as a doctor, nurse, relative, or organization responsible for managing the patient's care. In fact, the platform 16 can also communicate with the patient 50 through text message, telephone call, e-mail, or other communication modalities supported by the system.

This networked configuration enables flexible deployment of processing resources and supports real-time or near-real-time feedback to both patients and providers 48. It also facilitates integration with broader health monitoring systems and supports scalable data aggregation for population-level analytics or model refinement.

### Process for Various Embodiments

Illustrative embodiments use a multi-modal model to analyze physiological information collected from a patient. The model is configured to receive data from two or more distinct physiological modalities—such as weight, foot temperature, and fluid accumulation—each measured using physical sensors 50A or 50B. These sensors 50A and 50B may be integrated into a platform 16 or wearable device, such as those discussed above, which interacts directly with the patient's body. The model processes this sensor-derived data to identify patterns that may be indicative of heart failure decompensation.

The multi-modal model may be implemented using any of a variety of computational frameworks, including but not limited to rule-based logic, statistical inference, mathematical transformations, or machine learning algorithms. Improving on current technology, the multi-modal model preferably operates on temporally spaced data points, enabling it to detect changes or trends over time across multiple physiological domains. In some embodiments, the model is configured to correlate changes in one modality (e.g., weight gain) with changes in another (e.g., decreased foot temperature), thus identifying compound patterns that may not be apparent when analyzing each modality in isolation.

In some embodiments, the model is configured to analyze foot temperature data collected from at least one portion of the foot 10. For example, such embodiments may analyze foot temperatures collected from two or more spaced apart anatomical regions of the foot 10. These regions may include, for example, the heel, arch, metatarsal heads, hallux, or forefoot. By evaluating temperature changes across multiple locations (e.g., the same two or more locations at two different times) and weight data, the model can detect localized or asymmetric thermal patterns that may be indicative of impaired perfusion or early signs of decompensation. The model may compare temperature values of between these regions, or assess changes in each region over time, to identify compound or spatially distributed indicators of heart failure risk.

The model may be executed on a computing device that is local to the platform 16, remote from the platform 16, or distributed across multiple systems. It may operate in real time or on a scheduled basis, and may be configured to produce output information that supports clinical decision-making, patient monitoring, or automated alerts. The model's operation is rooted in the processing of physical measurements obtained from the patient, and its output is used to inform actions in the physical world, such as initiating a clinical intervention or prompting further diagnostic evaluation.

To those ends, the multi-modal model can be implemented specifically as a heart failure decompensation prediction model first derived from sensor data and other information from a set of one or more individual test subjects. This derivation data set includes individuals and/or periods of time in which an individual was not experiencing heart failure decompensation. It also includes episodes of heart failure decompensation as the detection or prediction target. Once derived, this model can be validated and deployed for monitoring.

The derivation process begins with signal acquisition/data collection, which involves gathering data from various temperature and weight sensors 50A and 50B, such as the platform 16, scale, and/or from an implanted cardiac monitoring device from test subjects. This data then may be combined with additional information sourced from databases, including one or more of leg and thoracic impedance, weight and weight changes, patient demographics, medical claim data, and adherence data. The historical data collected preferably encompasses a broad spectrum of outcomes for the variable of interest, which in this case includes heart failure decompensation. This comprehensive approach allows for a more accurate and detailed analysis of the patient's condition, facilitating better-informed healthcare decisions.

The process then normalizes and preprocesses the data. Specifically, to ensure accurate comparison, data may be preprocessed using techniques such as normalization, image processing, feature extraction, and/or noise reduction. For example:
i. Cleaning Data—remove or impute missing values, correct errors, handle outliers, perform extrapolation, interpolation, etc.
ii. Transforming Data—normalize or standardize data, particularly if variables are on different scales or if extreme values will bias analysis. If neural networks are planned for utilization, ensure data is encoded appropriately either through one-hot encoding or labeling. This also may involve normalizing the data to a standard coordinate system (e.g., enabling correspondence between contralateral locations on the feet).
iii. Partitioning Data—depending on method applied, split the data into training, testing, and validation sets for predictive modeling.
iv. Exploratory Data Analysis—build statistical summaries, review key metrics, and visualize the data prior any model training.

Next, the process selects a method, algorithm, and/or model to implement the identification of potential heart failure exacerbation. One technique may involve a simple sensitivity-adjusted threshold. Utilization of a sensitivity-adjusted threshold method may dynamically adjust a given threshold on the required sensitivity level. For example, in pattern recognition tasks, signals or patterns have varying degrees of strength, and sensitivity adjustments allow algorithms to be discerning about what is considered a significant event. To implement such a method, one skilled in the art may follow steps such as:
Threshold Setting—an initial threshold may be set based on historical data, domain knowledge, initial experimentation, or some combination of options.
   A weight change or change in water retention as measured by impedance over a duration of several days can indicate HF decompensation. A change of 5 pounds over 3 days is a typical threshold. However, weight change is non-specific and can be confounded by many other behaviors or conditions.
   Use foot temperature to adjust a threshold for weight change to predict HF.
Adjusting Sensitivity/Specificity—in this scenario, it is preferable to retain high-sensitivity given the consequences of missed events. Therefore, illustrative embodiments may retain a low threshold to detect a higher volume of signals, which will result in a larger volume of false positives than could be achieved via lower sensitivity monitoring. As an example, if the foot temperature is low for a given scan, the weight change sensitivity may be adjusted to a reduced threshold. Depending on preferences for mitigating false negatives or false positives, optimization can occur for sensitivity or specificity.
Application—incoming data may be assessed against the threshold to determine the next correct action for further processing.
   Compare weight change to the sensitivity-adjusted threshold to determine if an output should be generated.
   Example: a low foot temperature scan combined with an above-normal change in weight may trigger an action recommendation that could be routed to a clinical professional for purposes of engaging the patient and delivering needed care prior to exacerbation.

Various embodiments may employ any of a number of statistical methods. Statistical methods to consider include:

1. Probability Estimation—Determines the Likelihood of Different Outcomes Based on Historical Data. This May Involve One or More of:
Model Selection—parametric for normal distribution.
Parameter Estimation—calculate the parameters of the selected model using methods such as maximum likelihood estimation (MLE), Bayesian estimation, least squares estimation, etc.
Probability Calculation—calculate the probability of observing specific outcomes, again, probability of heart failure decompensation is the variable of interest.
Validation—compare predicted probabilities with actual outcomes in historical dataset.
Application—once the model is tuned, assess incoming data via the model, and for statistically significant probability of the variable of interest occurrence, generate an output indicating the risk of heart failure decompensation. Provide this output information to the user (patient), caretaker, remote monitoring service provider, and/or healthcare provider 48 so that intervention may be initiated to avoid further complication. Prompt an action for intervention.

### 2. Anomaly Detection

An anomaly detection method first may establish a model that defines “normal” using either parametric or non-parametric methods. A method may define anomalies utilizing statistical thresholds, e.g., standard deviation or quantiles. Any data point beyond three standard deviations from the mean can be considered an anomaly. The model may be applied to test data and calculate Z-scores to determine the number of standard deviations a given data point is from the mean. Detected anomalies from the prior step thus may be analyzed to confirm outliers are significant. Thresholds may be reevaluated as needed or baselines shift over time.

3. Smoothing and moving averages (preferable for univariate data)—technique for reduction of noise and identifying trends. This method may involve window size selection—select the appropriate number of data points (windows) to average in each calculation and then complete an average calculation per window generating a series of averages. The method then may smooth; e.g., the plot may average to visualize the smoothed trend, reducing noise. The method then analyzes trends, seasonality, forecasting opportunities, etc. One embodiment may look for temperature and weight baselines on a per patient basis, as well as look to reduce the noise of ambient temperature fluctuations, or anomalous scans. In the same vein, this method suits the weight gain criteria well, as the appropriate window size would allow for a tight indication, combined with temperature loss, of weight changes signaling cardiac exacerbation.

As noted, various embodiments use mathematical models. Those skilled in the art may select one or more of the following:

1. Logistic Regression. To this End, the Method/Model May Determine an Appropriate Standard Equation Utilized by Logistic Regression Models, and Train to Fit the Model to the Training Data. This May Involve the Following:
a. Initialization
b. Optimization—use an algorithm such as gradient descent to find the parameter values that minimize the cost function, typically binary cross-entropy loss:

Loss=−[y log(p(x))+(1−y)log(1−p(x))] 1.
c. Evaluation—this may predict, label (apply threshold) and review metrics, e.g., accuracy, precision, recall, F1-score, ROC.
d. Tuning—adjust feature selection, transformations, and/or model specification,
e. Deploy—implement tuned model into production system where outputs may be monitored and acted upon,
f. Feedback Loop—continue to feed new data into the model for ongoing training and to avoid model drift.

### 2. Bayesian Techniques

a. Prior Distribution Specification-illustrative embodiments may utilize empirical priors given information available on the relationship between foot temperature and cardiac output. Those priors may include one or more of:
   Conjugate Priors-used for simplification of the analysis as they yield posterior distributions that are of the same family as the prior,
   Noninformative Priors-exert minimal influence on the posterior,
   Empirical Priors-informed by historical data.
b. Likelihood Function Selection-given the data collected, it is probable that a Normal Distribution, Poisson Distribution, Exponential Distribution, and/or Multinomial Distribution may all be good candidates for the likelihood function during the model build phase.
c. Posterior Distribution Compute-combine prior distribution with likelihood function to calculate the posterior distribution.
d. Evaluation, which may involve:
   Convergence Diagnostics—determine if methods selected have converged (e.g., Monte Carlo, Markov Chain, etc.),
   Predictive Checks—generate predictions and compare to actuals to evaluate model fit,
   Sensitivity Analysis-adjust the priors to evaluate sensitivity to changes in assumptions.

### 3. Hidden Markov Models (HMM)

a. Model Specification.
   Define States—determine the number and nature of hidden states in the model,
   Define Observations—identify the possible observable outputs linked to each state,
   Parameter Initialization—initialize the transition probabilities between states, the emission probabilities of observations given states, and the initial state probabilities.
b. Parameter Estimation
   Expectation-Maximization technique—use this technique/method to find the unknown parameters (transition and emission probabilities) of the HMM by iteratively estimating probabilities until convergence is achieved,
   Forward-Backward Algorithm—calculates the probabilities of being in a particular state at a particular time given the sequence of observations.
c. Model Training—adjust probabilities in the model to maximize the likelihood of the observed sequences.
Ensure convergence to a stable solution,
Cross-validate.
d. Decoding & Prediction
Viterbi Algorithm—find the most likely sequence of hidden states given the observed data. For example, some embodiments may utilize this model to identify underlying patterns in the weight data combined with temperature via thermometry, to predict potential cardiac events as an identified sequence. Another option may use the HMM to predict the probability of the next observation, i.e., scan results, which could then be extrapolated to a further number of future scans.

### 4. Machine Learning Technology.

a. Bernoulli Naïve Bayes (BNB), which involves model training.
   Calculate Prior Probabilities—frequency of each class in the training dataset,
   Calculate Likelihoods—determine conditional probabilities for all classes by counting frequency of each feature per label, and apply smoothing.
   Evaluation—using the test dataset, calculate posterior probabilities for each class and identify the class with the highest probability per metrics like accuracy, precision, specificity, recall, etc.
   Prediction—ensure viability of model utilizing test and validation datasets to predict probabilities for each class.

### 5. K-Nearest Neighbors (KNN).

a. K-determination—select the appropriate number of neighbors.
   i. A smaller k is preferable in this scenario though it may lead to higher levels of false positives.
b. Distance Metric—choose the appropriate distance metric to measure distances between data points.
   i. While each metric should be vetted, likely the Euclidean distance will be selected due to simplicity and fit to the data.
c. Prediction
   i. Calculate distance from new data to all points included in training dataset,
   ii. Identify nearest neighbors based on the Euclidean distance,
   iii. Aggregate and classify the new data point based upon nearest neighbors.
d. Evaluate utilizing metrics e.g., accuracy, precision, specificity, recall, etc.

### 6. Support Vector Machines (SVM)

a. Hyperparameter Selection
   i. Regularization (C)—optimizing parameter for training error and model complexity. In our scenario, this needs to be a relatively high value of C,
   ii. Kernel—linear, polynomial, RBF, etc.
      1. An SVM using an RBF kernel may be particularly helpful in image processing of scan transmitted,
   iii. *Gamma—if a non-linear kernel is selected, a gamma value will need to be selected.
b. Model Training,
c. Evaluate utilizing metrics e.g., accuracy, precision, specificity, recall, etc.,

### 7. Random Forest Technology

a. Hyperparameter Selection
   i. Determine the number of trees, balancing reduction of overfitting with cost of compute,
   ii. Maximum depth of trees, again balancing overfitting with identification of complex patterns,
   iii. Set the minimum sample split threshold to create a new node,
   iv. Minimum number of samples required to be at a leaf node.
b. Train the model via decision tree generation by bootstrapping.
c. Evaluate utilizing metrics e.g., accuracy, precision, specificity, recall, etc.

### 8. Neural Networks

a. Multi-Layer Perceptron (MLP)
   i. Architecture Determination
      1. Input layer size should match the number of features in the data,
      2. Determine the number of hidden layers and number of neurons per layer. A relatively small number of layers and/or neurons will be preferable for our data,
      3. Choose an activation function, e.g., Rectified Linear Unit, softmax, sigmoid, etc.,
      4. Depending on activation function and the data problem, define your output layer. We will have one neuron in the output given our binary classification problem and sigmoid activation function.
   ii. Model Compilation
      1. Determine the loss function (cross-entropy for classification).
      2. Select an optimizer to minimize the loss function by updating the weights.
   iii. Ensure feed-forward and backpropagation during training
   to update weights, minimize loss, and reduce errors.
b. Model Tuning—regardless of method selected, each model will need to have parameters tuned, assumptions tested, values adjusted, etc. to optimize performance. The sensitivity and specificity of the model are optimized to provide the favorable results for the care model of the patient.
c. Validation—The model is then validated against similar sets of patient sensor and health data that were not used in the derivation or training set to determine its predicted accuracy when deployed.
d. Implementation/Deployment—once the model(s) has been trained and is performing effectively, it can be implemented into a core system to provide predictions, inform data, trigger intervention, or some combination of these actions. The method for deployment will vary dependent upon the underlying architecture and infrastructure, but two examples could be:
   i. API call—ensure trained model is serialized or pickled and exposed as a service for a system with the appropriate endpoint can call the API,
   ii. Sequential step—add the trained model into a broader data flow, so that following processing, output data flows directly into the model as input, predictions are generated, and said predictions are added along with the data used as output into an accessible system, e.g., database,
   iii. Evaluation/Feedback—signals that are identified above the threshold are utilized to drive interventions, and outcomes are fed back into the model for ongoing sensitivity/specificity support, at the individual level.

FIG. 6A schematically shows components of a system for processing patient data to develop a risk assessment for heart failure decompensation. Each of these components is operatively connected by any conventional interconnect mechanism. It should be noted that FIG. 6A only schematically shows each of these components. Those skilled in the art should understand that each of these components can be implemented in a variety of conventional manners, such as by using hardware, software, or a combination of hardware and software, across one or more other functional components. For example, the components may be implemented using a plurality of microprocessors executing firmware, as one or more application specific integrated circuits (i.e., “ASICs”) and related software, and/or a combination of ASICs, discrete electronic components (e.g., transistors), and microprocessors. Accordingly, the representation of a component in a single box is for simplicity purposes only. In fact, in some embodiments, one ore more of the shown components is distributed across a plurality of different machines—not necessarily within the same housing or chassis.

Moreover, some components are not shown for simplicity. Thus, those skilled in the art should understand that such a device has many other physical and functional components, such as central processing units, other processing modules, and short-term memory. Accordingly, this discussion is in no way intended to suggest that FIG. 6A represents all the elements.

As shown, the system of this embodiment includes the platform 16 (e.g., an open or closed platform 16), which includes one or more of the noted temperature sensors 50A, body weight sensors 50B, body composition sensors, and a number of other items. This platform 16 may be considered in part as a data collection device, which collects and forwards relevant information to a data collection and storage system, which may be on the platform 16 or at a remote location. A model analyzer (e.g., an analysis engine 46) processes this information, as described herein, and produces an output message with information relating to the risk heart failure decompensation via an output interface.

FIG. 6B schematically shows another related embodiment of this system, showing the platform 16 in the network 44 (FIG. 5) with its interconnected components in more detail. As shown, the patient communicates with the platform 16 by communicating temperature, bioimpedance, weight, or other via the sensor(s) 50A, 50B, and other sensors. A data acquisition engine 54, implemented by, for example, a motherboard and circuitry, controls acquisition of the data for storage in a storage device 56. Among other things, the storage device 56 can be a volatile or nonvolatile storage medium, such as a hard drive, high-speed random-access-memory (RAM), and/or solid-state memory. The input/output interface port 58, also controlled by the motherboard and other electronics on the platform 16, selectively transmits or forwards the acquired data from the storage device 56 to the analysis engine 46 on a remote computing device, such as a server 60. The data acquisition engine 54 also may control the user indicators/displays 18, which provide feedback to the user through the above mentioned indicia (e.g., audible, visual, or tactile).

The analysis engine 46 on the remote server 60 analyzes the data received from the platform 16 in conjunction with a health data analytics device 62. Among other things, the health data analytics module may incorporate historical patient data, population-level trends, or clinical thresholds to contextualize the incoming measurements and refine the model's output. It may also apply statistical or machine learning techniques to enhance risk stratification and support longitudinal tracking. A server output interface 64 forwards the processed output information/data from the analysis engine 46 and health data analytics module toward others across the network 44, such as to a provider 48, a web display, or to the user via a phone, e-mail alert, text alert, or other similar way.

This output message may have the output information in its relatively raw form for further processing. Alternatively, this output message may have the output information formatted in a high-level manner for easy review by automated logic or a person viewing the data. Among other things, the output message may indicate risk of heart failure decompensation, the state of heart failure, or other related information.

Using a distributed processing arrangement like that shown in FIG. 6B has a number of benefits. Among other things, it permits the platform 16 or modality to have relatively simple and inexpensive components that are unobtrusive to the patient. Moreover, this permits a “software-as-a-service” business model (“SAAS model”), which, among other things, permits more flexibility in the functionality, typically easier patient monitoring, and more rapid functional updates. In addition, the SAAS model facilitates accumulation of patient data to improve analytic capability.

Some embodiments may distribute and physically position the functional components in a different manner. For example, the platform 16 (e.g., the thermal camera, open platform 16, or closed platform 16) may have the analysis engine 46 on its local motherboard. In fact, some embodiments provide the functionality entirely on the modality, such as on the open platform 16 and/or within other components in the local vicinity of the platform 16. For example, all of those functional elements (e.g., the analysis engine 46 and other functional elements) may be within a housing that also contains a thermal or standard non-thermal camera. Accordingly, discussion of a distributed platform 16 is but one of a number of embodiments that can be adapted for a specific application or use.

Those skilled in the art can perform the functions of the analysis engine 46 (and the other functional modules) using any of a number of different hardware, software, firmware, or other non-known technologies. For example, the analysis can include the multi-modal model described herein to process the health information.

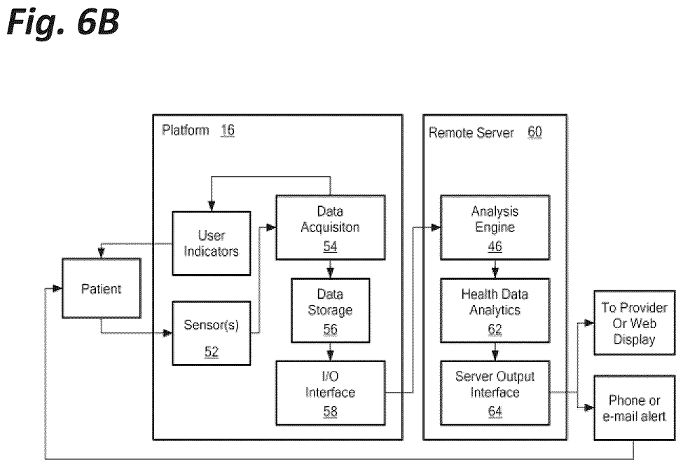

Illustrative embodiments also may use the process of FIG. 7. It should be noted that like the other processes described in this document, this process is simplified from a longer process. Accordingly, the process of may have other steps that those skilled in the art likely would use. In addition, some of the steps may be performed in a different order than that shown, or at the same time. Those skilled in the art therefore can modify the process as appropriate. Moreover, as noted above and below, many of the processes and structures noted are but one of a wide variety of different processes and structures that may be used. Those skilled in the art can select the appropriate processes and structures depending upon the application and other constraints. Accordingly, discussion of specific materials and processes is not intended to limit all embodiments.

The process uses the analysis engine 46 to analyze foot temperature, which can be conducted using a systematic sub-process having a plurality of steps. To that end, the first step 700 involves measuring foot temperature on the open platform 16. This can be done by taking a comprehensive measurement across part of or the entire foot 10, followed by focusing on specific anatomical locations, such as the arch or hallux, or regions like the metatarsal heads or forefoot. These areas of interest can be pinpointed either manually or with the assistance of a trained machine vision model. Alternative embodiments may take temperature measurements of portions of the foot 10. Yet other embodiments may form a thermogram of at least a portion of the foot 10.

After the data is collected, the next step determines a representative temperature for the whole foot 10, which could be the average or median temperature across some or all measurements. Alternatively, discrete temperature sensors 50A may be strategically placed at predetermined locations to gather targeted data.

In parallel, the ambient temperature may also be recorded to provide context to the foot temperature readings. This can be achieved using the same temperature sensor(s) 50A on the platform 16 or by employing a separate temperature sensor 50A designed for environmental readings.

Finally, the sub-process compares or normalizes the foot temperature against a baseline. This may involve adjusting the foot temperature by subtracting the ambient temperature to calculate the foot temperature above ambient temperature. Additionally, comparisons can be made between different anatomical regions of the foot 10, such as subtracting the arch temperature from the toe temperature, or comparing the toe temperature to the maximum, average, or median foot temperature. These comparisons help in normalizing the data and providing a clearer picture of the foot's thermal profile.

After, before, or while analyzing foot temperature, the process may analyze weight change. To that end, at step 702, the process may measure the patient's weight using the open platform 16. This should be done consistently, either daily or at multiple time points throughout the day, to ensure accuracy and reliability. The measurements should be recorded and compiled into a series, which will serve as a comprehensive record of the patient's weight over time.

From this series of weight measurements, an earlier weight, such as a baseline, can be established. This baseline may represent the patient's average or typical weight over a defined period and serves as a reference point against which future measurements can be compared. Establishing a baseline helps account for natural fluctuations in weight and enables detection of significant deviations. In other embodiments, the system may instead use one or more prior weight measurements without computing a formal baseline.

The current weight measurement then may be compared to the baseline. This comparison can be done by subtracting the current weight from the baseline weight to calculate the difference. This difference, whether it is a gain or loss, is indicative of a change in the patient's weight status. Monitoring these changes over time is important for detecting potential health issues, assessing the effectiveness of dietary or lifestyle interventions, and making informed decisions about the patient's care.

The process then may transmit the temperature and/or weight data to a processor, controller, or other device (e.g., a computer) at step 706. Among other things, this processor can either be integrated within the measurement device, located remotely, or distributed locally and remotely.

If the processor is in the measurement device, may be is stored locally (step 704) where the processor can aggregate and analyze the data over time. This allows for rapid processing and can be beneficial for quick assessments and real-time monitoring. The local storage 56 and processing ensure that data is readily available for analysis without the need for external transmission, which can be advantageous in situations where network connectivity is limited, or security is a concern.

Alternatively, when the processor is remote, the apparatus transmits the data to this remote processor for analysis (step 708). This transmission can occur over the network 44 to a remote server, which allows for more robust data analysis and storage capabilities. Additionally, the data can be sent from the apparatus to another device, such as a smartphone, tablet, or computer, which then relays the information to the remote processor. This method is particularly useful for long-term tracking and analysis, as it enables the data to be stored and processed on powerful servers that can handle large datasets and complex computations.

Regardless of the location of the processor, the goal is to ensure that the data is accurately and securely transmitted for analysis. The choice between a local or remote processor will depend on the specific requirements of the monitoring system, including considerations of immediacy, connectivity, security, and the complexity of the data analysis needed.

Then, at step 710, the analysis engine 46 with the model, applies temperature and weight change to the above discussed heart failure prediction model. Specifically, to apply foot temperature and weight change to a heart failure prediction model (the multi-modal model), several approaches can be utilized, each offering a unique methodology to enhance the accuracy of heart failure risk assessment.

In the sensitivity-adjusted model, the process begins with the measurement and normalization of foot temperature to estimate cardiac output. Cardiac output is correlated with foot temperature, where higher foot temperatures indicate better cardiac output. The estimation of cardiac output can be achieved through linear or non-linear calculations, or it may be stratified into multiple tiers, such as high, medium, and low. Following this, body weight change compared to a baseline measurement is assessed. The threshold for predicting the risk of heart failure decompensation may then be adjusted based on the weight change, taking into account the normalized foot temperature.

Specifically, if the normalized foot temperature is high, indicating good or high cardiac output, the weight change threshold may be adjusted higher, meaning a larger increase in weight over the baseline is required to indicate a risk of heart failure decompensation. Conversely, if the normalized foot temperature is low, indicating low cardiac output, the weight change threshold may be adjusted lower, meaning a smaller increase in weight over the baseline is sufficient to indicate a risk of heart failure decompensation. Accordingly, this approach may dynamically adjust thresholds based on readings from other concurrently, prior, and/or subsequently sensed physiological parameters.

Alternatively, a statistical model can be employed. This approach also starts with measuring and normalizing foot temperature. A statistical model is then applied to determine the probability that this measurement contains an anomaly or is correlated with heart failure risk. Similarly, body weight is measured and compared to the baseline, with another statistical model applied to assess the probability of an anomaly or correlation with heart failure risk. The risk probabilities derived from these statistical estimations are combined, and their intersection determines the overall probability that the user is experiencing heart failure decompensation.

Another approach involves a mathematical model, which involves measuring and normalizing foot temperature, followed by measuring body weight and comparing it to the baseline. Both normalized measurements are then included in the mathematical model, either as a single measurement at a point in time or as a series where the model estimates the risk based on the progression of values over time. The mathematical model then calculates the risk of the user experiencing heart failure decompensation.

As another alternative, some embodiments may use a machine learning algorithm. This method begins with the measurement and normalization of foot temperature, followed by measuring body weight. These normalized measurements are then included in the machine learning algorithm. The algorithm incorporates the new measurements into a series of historical measurements to calculate an estimate of the user's risk of heart failure decompensation. This approach allows the algorithm to continually learn and adjust its risk estimations based on the latest data.

Each of these types of models—sensitivity-adjusted, statistical, mathematical, and machine learning—provides a structured approach to integrating foot temperature and weight change measurements into heart failure risk prediction, thereby enhancing the accuracy and reliability of early heart failure decomposition detection and intervention.

Step 712 then generates output information indicating the user's risk of heart failure decompensation. To be effective, this output should communicate information relating to the assessed risk in a format that is accessible, actionable, and tailored to the user's needs. The output may be delivered directly to the user through various channels, such as the noted display 18 on the measurement device itself, a mobile application, or a connected health system. Immediate feedback on the device—such as visual indicators, numeric scores, or alerts—can prompt the user to take timely action, such as contacting a healthcare provider 48 or adjusting behavior. Preferably, these and other transmissions are encrypted to ensure data security and privacy.

The information may also be integrated into a dedicated application installed on the user's smartphone or tablet. Such an application may provide a user-friendly interface with personalized insights, historical comparisons, and educational content to support self-management. For users who prefer traditional communication methods, the output may be included in a printed report, email, or secure message, ensuring flexibility in how results are delivered and acted upon.

In addition to informing the user, the output may be transmitted to a healthcare provider 48, caregiver, or remote monitoring service to support clinical oversight and timely intervention. Communicating the user's risk of heart failure decompensation to a healthcare provider 48 enables more effective monitoring and facilitates informed decision-making regarding treatment plans. The output may be displayed through a specialized application designed for medical professionals, which can integrate with existing electronic health record (EHR) systems to provide seamless access to the user's risk data alongside their broader medical history.

Alternatively, the risk assessment may be delivered through secure channels such as encrypted email, fax, or a dedicated clinician portal, ensuring timely and accurate delivery regardless of the provider's technological infrastructure. In some embodiments, the output may also trigger automated workflows, such as scheduling follow-up assessments, initiating telehealth consultations, or logging events in the EHR. The output may include trend data, confidence scores, or contextual explanations to support interpretation and clinical decision-making by both patients and providers 48.

Integration of risk assessments into a comprehensive healthcare management system can enhance collaboration between users and their healthcare providers 48. By sharing real-time data and insights, users can receive personalized advice and interventions, while healthcare providers 48 can track progress and adjust treatment plans, as necessary. This collaborative approach not only improves the quality of care but also empowers users to take an active role in managing their heart health.

Some or all of the analysis and management steps in this process may be performed by the analysis engine 46, which may operate locally, in a distributed manner, or remotely. In various embodiments, the analysis engine 46 executes the multi-modal model, evaluates trends in the physiological data, and generates output information for clinical decision-making or patient monitoring.

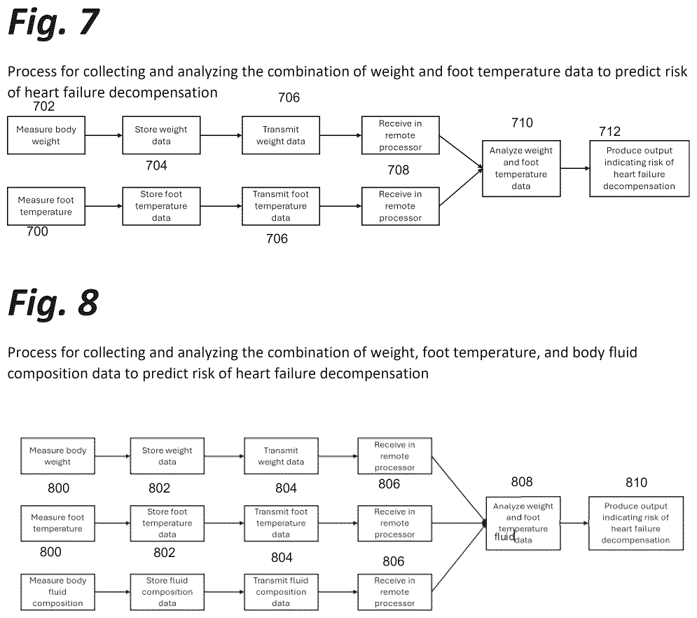

FIG. 8 shows a process of predicting the risk of heart failure decompensation using weight, foot temperature, and/or body fluid composition data in accordance with illustrative embodiments. The process begins by receiving and measuring weight, body fluid composition, and foot temperature using a sensor-equipped platform 16 (step 800). This platform 16 may be an open platform 16, such as that shown in FIG. 1, or a closed platform 16, such as a wearable insole or sock. These platforms 16 include one or more of weight sensor(s) 50B (e.g., load cells, strain gauges, or pressure-sensitive elements) and temperature sensor(s) 50A (e.g., thermistors, thermocouples, or infrared detectors) positioned to capture data from multiple anatomical regions of the foot 10. In some embodiments, the platform 16 also includes bioimpedance sensors configured to assess body fluid composition, such as extracellular and intracellular water content, which may be indicative of fluid retention associated with heart failure.

The data collected from these sensors may include temporally spaced measurements of weight, temperature, and fluid accumulation (e.g., information obtained at an earlier time). These measurements may be taken at regular intervals or in response to specific events, and may be associated with baseline values or prior readings to enable longitudinal (i.e., temporal) comparison. The data is stored locally on the platform 16 or transmitted—either directly or via an intermediary device such as a smartphone or tablet—to a designated processor and/or remote storage device 56. This processor may be located locally (e.g., on the platform 16 or a nearby device), remotely (e.g., on a cloud server), or distributed across multiple systems.

Once received, the data is stored (step 802) and transmitted to the remote server 60 (step 804). After received at step 806, the analysis engine 46 analyzes the received data using a multi-modal model configured to evaluate the combined physiological information (step 808). As noted above, the model may be implemented using rule-based logic, statistical inference, mathematical transformations, or machine learning algorithms. It may operate on raw sensor data, pre-processed values, or derived metrics, and may incorporate temporal comparisons to detect trends or deviations over time. In some embodiments, the model evaluates whether changes in weight, temperature, and/or fluid composition collectively indicate a physiological pattern associated with heart failure decompensation. The model may also incorporate contextual inputs such as ambient temperature, patient-reported symptoms, or historical health data.

Next, at step 810, the method produces output indicating the risk of heart failure decompensation. The output of the model is not merely a collection of raw data points, but a refined interpretation that indicates the potential risk of heart failure decompensation. In some embodiments, the output may include an assessment of overall heart failure risk. Either way, the output may take the form of a binary classification (e.g., risk/no risk), a numerical risk score, or a qualitative assessment (e.g., low, moderate, or elevated risk). The output may be displayed on the platform 16 itself, transmitted to a mobile application, or integrated into an electronic health record (EHR) system. It may also be used to trigger automated workflows, such as scheduling follow-up assessments, initiating telehealth consultations, or notifying a healthcare provider 48.

Alternative embodiments may focus on a subset of the available data. For example, some implementations may omit weight analysis and rely solely on foot temperature and fluid retention data to assess risk. This flexibility allows the system to adapt to different use cases, sensor configurations, and patient populations. Regardless of the data used, the process is configured to provide a clinically meaningful assessment that supports early detection, timely intervention, and improved management of heart failure.

As with the process of FIG. 7, some or all of the analysis and management steps in this process may be performed by the analysis engine 46, which may operate locally, in a distributed manner, or remotely. Accordingly, in various embodiments, the analysis engine 46 executes the multi-modal model, evaluates trends in the physiological data, and generates output information for clinical decision-making or patient monitoring.

In various embodiments, including those of FIGS. 7 and 8, the system may incorporate additional patient-reported information to enhance the accuracy and contextual relevance of the output message regarding heart failure risk or status. These embodiments may enable a more comprehensive assessment by integrating subjective symptom data alongside objective physiological measurements.

For example, the system may prompt the patient with a series of targeted questions designed to elicit key indicators of decompensated heart failure, such as:
Paroxysmal Nocturnal Dyspnea (PND): “Are you waking in the middle of the night to catch your breath?”
Orthopnea: “How many pillows are you using to sleep? Are you sleeping in bed or somewhere else?”
Fatigue: “Has your energy level decreased? Are you tired or sluggish? Are you unable to do the things you normally do?”
Shortness of Breath: “Are you feeling more short of breath than usual?”
Edema: “Are your legs more swollen this morning than normal?”
Palpitations: “Have you had any different beating of your heart?”
Abdominal Swelling: “Is your stomach swollen? Are your pants more snug than usual?”
Nausea: “Are you feeling sick to your stomach?”
Chest Pressure or Discomfort: “Are you experiencing any chest tightness, pressure, or discomfort?”
Unexplained Weight Gain: “Have you gained weight unexpectedly over the past few days?”
Reduced Urine Output: “Have you noticed a decrease in how often or how much you're urinating?”
Cognitive Changes or Confusion: “Have you felt more confused, forgetful, or mentally foggy than usual?”
Loss of Appetite or Early Satiety: “Are you eating less than usual or feeling full quickly?”

The multi-modal model of the analysis engine 46 processes this subjective input/answers in conjunction with the noted objective data streams, such as daily weight, foot temperature, fluid accumulation, and other sensor-derived metrics. For example, these answers may be incorporated into the model as categorical inputs (e.g., binary or ordinal values), which are processed alongside sensor-derived physiological data. The model may assign predefined weights to these inputs based on clinical relevance or learned associations from training data. For example, a reported increase in shortness of breath may increase the model's sensitivity to concurrent changes in weight or temperature. In other embodiments, the model may treat these responses as modifiers that adjust thresholds or influence the interpretation of sensor data. This integration of subjective and objective inputs allows the model to generate a more context-aware and individualized risk assessment.

As another example, a modest increase in weight might not trigger an alert on its own, but when paired with reported orthopnea and elevated foot temperature, the model may classify the patient as high risk for decompensation. This integrative approach allows the system to deliver more personalized and timely alerts, improving early detection and enabling proactive clinical intervention.

To support the predictive capabilities of the system, preliminary experimental data were collected and analyzed from patients who were later hospitalized for heart failure. These data provide empirical support for the physiological trends captured by the system's multi-modal model, particularly in relation to changes in peripheral temperature and body weight.

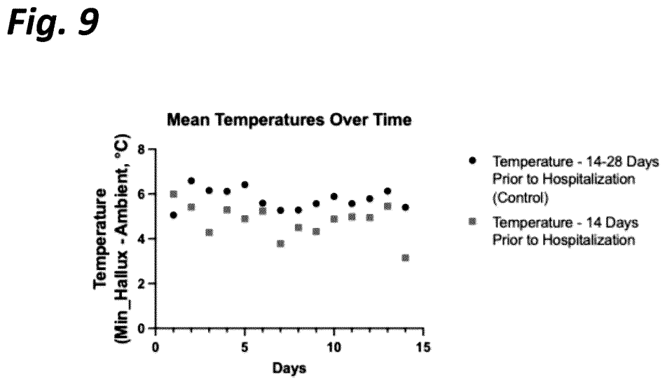

In one embodiment, the system leverages temperature differentials—specifically, the minimum hallux (big toe) temperature minus ambient temperature—as a proxy for peripheral perfusion. Aggregate patient data demonstrate a consistent decline in this temperature differential in the 14 days leading up to hospitalization. This trend is visualized in FIG. 9, which shows mean temperature values over time, with dots representing the 14-28 day pre-hospitalization window and boxes representing the critical 0-14 day window. The narrowing of standard error bars in the latter period suggests a convergence of physiological deterioration across patients, reinforcing the model's ability to generalize risk detection.

Additionally, individual case studies further illustrate the model's utility. A 54-year-old male with New York Heart Association Class III symptoms exhibited a steady decline in toe temperature alongside increasing weight and water mass over the month preceding hospitalization. Similarly, a 29-year-old male who presented with cardiogenic shock showed a marked drop in toe temperature and concurrent weight gain. These cases exemplify the system's ability to detect early signs of decompensation through combined thermal and weight monitoring.

Importantly, these findings validate the system's approach of integrating sensor-derived metrics with patient-reported symptoms to identify heart failure phenotypes. The model's classification schema—such as “warm and wet” or “cold and dry”—is informed by both traditional clinical frameworks and novel digital biomarkers, enabling more nuanced and timely risk stratification.

In one example, the multi-modal model is implemented as a feedforward neural network trained to predict the likelihood of heart failure decompensation based on two primary inputs: (1) the change in body weight over a defined time window, and (2) the change in foot temperature, normalized against ambient temperature. The model receives temporally spaced measurements of both parameters—such as daily readings over a 14-day period—and computes the difference between the most recent value and a baseline average. These two features are input into a neural network consisting of an input layer, one or more hidden layers with non-linear activation functions (e.g., rectified linear unit), and a final output layer with a sigmoid activation function that produces a probability score between 0 and 1.

The model is trained using labeled historical data from patients with known decompensation events. During training, the network minimizes a binary cross-entropy loss function using an optimization algorithm such as stochastic gradient descent or Adam. The training process includes validation and tuning of hyperparameters such as learning rate, number of hidden units, and regularization strength. Once trained, the model is deployed to the remote server 60 (or an edge device) where it receives real-time sensor data from the patient's platform 16. Using the analysis engine 46, the server 60 applies the trained weights to the incoming data and generates a risk score, which is compared to a predefined threshold to classify the patient's risk level. This output is then transmitted to a clinician-facing dashboard or patient-facing application for review and potential intervention.

Accordingly, various embodiments illustrate a technical solution using a data-centric system for early detection of heart failure decompensation that addresses limitations in conventional monitoring approaches. Rather than relying on episodic clinical assessments or isolated metrics, the system integrates multiple physiological and behavioral inputs into a unified framework that supports individualized, longitudinal risk assessment.

To that end, the analysis engine 46 with the multi-modal model, which processes heterogeneous data (including peripheral temperature trends, weight fluctuations, and structured symptom responses), uses algorithmic logic to detect clinically meaningful deviations from baseline or earlier data. This model and analysis engine 46 evaluate temporal patterns and cross-signal correlations, enabling the system to identify subtle but converging indicators of decompensation. For example, the analysis engine 46 may detect a combination of modest weight gain, declining foot temperature, and reported orthopnea as a high-risk configuration, even if each signal alone would not meet a predefined threshold.

This architecture reflects a technically grounded approach to clinical decision support. By transforming raw, time-series inputs into structured, interpretable outputs through defined computational processes, the system enables timely and context-aware alerts. These capabilities illustrate how integrated modeling and real-time analysis can enhance the precision and responsiveness of heart failure management in both home and clinical settings.

Various embodiments of the invention may be implemented at least in part in any conventional computer programming language. For example, some embodiments may be implemented in a procedural programming language (e.g., “C”), or in an object-oriented programming language (e.g., “C++”). Other embodiments of the invention may be implemented as a pre-configured, stand-alone hardware element and/or as preprogrammed hardware elements (e.g., application specific integrated circuits, FPGAs, and digital signal processors), or other related components.

In an alternative embodiment, the disclosed apparatus and methods (e.g., see the various flow charts described above) may be implemented as a computer program product for use with a computer system. Such implementation may include a series of computer instructions fixed either on a tangible, non-transitory medium, such as a computer readable medium (e.g., a diskette, CD-ROM, ROM, or fixed disk). The series of computer instructions can embody all or part of the functionality previously described herein with respect to the system.

Those skilled in the art should appreciate that such computer instructions can be written in a number of programming languages for use with many computer architectures or operating systems. Furthermore, such instructions may be stored in any memory device, such as semiconductor, magnetic, optical, or other memory devices, and may be transmitted using any communications technology, such as optical, infrared, microwave, or other transmission technologies.

Among other ways, such a computer program product may be distributed as a removable medium with accompanying printed or electronic documentation (e.g., shrink wrapped software), preloaded with a computer system (e.g., on system ROM or fixed disk), or distributed from a server or electronic bulletin board over the network (e.g., the Internet or World Wide Web). In fact, some embodiments may be implemented in a software-as-a-service model (“SAAS”) or cloud computing model. Of course, some embodiments of the invention may be implemented as a combination of both software (e.g., a computer program product) and hardware. Still other embodiments of the invention are implemented as entirely hardware, or entirely software.

The embodiments described above are intended to be merely exemplary; numerous variations and modifications will be apparent to those skilled in the art. Such variations and modifications are intended to be within the scope of various embodiments.

Some embodiments may be implemented with the following innovations:
1A. A heart failure management system comprising:
   an input configured to receive multi-modal physiological information from a platform, the platform having at least one temperature sensor configured to produce foot temperature information associated with the temperatures of two or more spaced apart portions of the patient's foot, the platform further having at least one fluid sensor configured to produce patient fluid accumulation information, the platform including at least one of an open platform and a closed platform;
   an analysis engine with a multi-modal model configured to analyze physiological information received at the input, the analysis engine configured to receive first foot temperature information produced by the at least one temperature sensor, the model also configured to receive earlier foot temperature information of the patient, the first foot temperature information being temporally spaced from the earlier foot temperature information, the first foot temperature information and earlier foot temperature information of the patient each associated with two or more portions of the patient's foot,
   the analysis engine further configured to receive first fluid accumulation information and earlier fluid accumulation information of patient, the first fluid accumulation information being temporally spaced from the earlier fluid accumulation information, the multi-modal model of the analysis engine configured to determine whether 1) the first fluid accumulation information, 2) the earlier fluid accumulation information, 3) the first foot temperature information, and 4) the earlier foot temperature information collectively shows a pattern indicative of heart failure decompensation; and
   an output configured to produce output information for use in clinical decision-making or patient monitoring relating to whether the multi-modal model determined a pattern indicative of heart failure decompensation.
2A. The system of innovation 1A further comprising the platform.
3A. The system of innovation 2A wherein the platform comprises an open platform.
4A. The system of innovation 3A wherein at least a portion of the analysis engine is remote from the open platform.
5A. The system of innovation 1A wherein the analysis engine is configured to:
   determine whether the first fluid accumulation and earlier fluid accumulation indicate a change of fluid accumulation; and
   determine whether the first foot temperature information and the earlier foot temperature information indicate a change in foot temperature,
   the model using the determinations of both change of fluid accumulation and change of foot temperature to determine whether 1-4 collectively indicates heart failure decompensation.
6A. The system of innovation 1A wherein the first foot temperature information are associated with a given two or more portions of the patient's foot, the earlier foot temperature information also are associated with the given two or more portions of the patient's foot.
7A. The system of innovation 1A further comprising a display to display output indicia relating to whether output information includes information relating to a pattern indicative of heart failure decompensation.
8A. The system of innovation 1A wherein the earlier foot temperature information comprises a representation of the temperature of the whole foot as a median or average temperature across measurements before the earlier temperature measurement.
9A. The system of innovation 1A wherein the model is configured to use weight information to determine whether a pattern indicative of heart failure decompensation exists.
10A. The system of innovation 9A where the at least one fluid sensor comprises at least one bioimpedance sensor.
11A. The system of innovation 1A wherein the model may include one or more of a sensitivity adjusted model, a statistical model, a mathematical model, and a machine learning technique.
12A. The system of innovation 1A further wherein the model is configured to receive answers from the patient from a plurality of questions, the model using at least one of the answers and items 1-4 to determine whether a pattern indicative of heart failure decompensation exists.
13A. A method of identifying a risk of heart failure decompensation of a patient, the method comprising:
   receiving first fluid accumulation information and earlier fluid accumulation information of the patient, the first fluid accumulation information being temporally spaced from the earlier fluid accumulation information;
   receiving first foot temperature information and earlier foot temperature information of patient, the first foot temperature information and earlier foot temperature information of the patient each associated with two or more portions of the patient's foot, the first foot temperature information being temporally spaced from the earlier foot temperature information,
   at least one of the first fluid accumulation information and the first foot temperature information being produced by and communicated from a platform having at least one fluid accumulation sensor to detect fluid accumulation information and at least one temperature sensor configured to determine the temperature at different spaced apart portions of the foot, the platform including one of an open platform and a closed platform;
   using a multi-modal model executing on at least one computing device and configured to analyze physiological data to determine whether 1) the first fluid accumulation information, 2) the earlier fluid accumulation information, 3) the first foot temperature information, and 4) the earlier foot temperature information collectively shows a pattern indicative of heart failure decompensation; and
   producing output information for use in clinical decision-making or patient monitoring relating to whether the model determined a pattern indicative of heart failure decompensation.
14A. The method of innovation 14A wherein using comprises:
   determining whether the first fluid accumulation information and earlier fluid accumulation information indicate a change of fluid accumulation; and
   determining whether the first foot temperature information and the earlier foot temperature information indicate a change in foot temperature,
   the model using the determinations of both change of fluid accumulation and change of foot temperature to determine whether 1-4 collectively indicates heart failure decompensation.
15A. The method of innovation 14A wherein the first foot temperature information are associated with a given two or more portions of the patient's foot, the earlier foot temperature information also are associated with the given two or more portions of the patient's foot.
16A. The method of innovation 14A further comprising displaying on a display device output indicia relating to whether the model determined a pattern indicative of heart failure decompensation.
17A. The method of innovation 14A wherein the earlier fluid accumulation information comprises a baseline of fluid accumulation information, further wherein the earlier foot temperature information comprises a baseline of temperature information.
18A. The method of innovation 14A wherein the earlier foot temperature information comprises a representation of the temperature of the whole foot as a median or average temperature across measurements before the earlier temperature measurement.
19A. The method of innovation 14A further comprising receiving past and present patient weight information relating to the patient, said using a model comprising also using the past and present patient weight information to determine whether a pattern indicative of heart failure decompensation exists.
20A. The method of innovation 14A wherein the at least one fluid accumulation sensor comprises bioimpedance sensors to determine fluid accumulation in the patient.
21A. The method of innovation 14A wherein receiving first fluid accumulation information comprises receiving the fluid accumulation information from an open platform having a fluid accumulation sensor.
22A. The method of innovation 14A wherein receiving foot temperature information comprises receiving the foot temperature information from an open platform having one or more temperature sensors.
23A. The method of innovation 14A wherein the model may include one or more of a sensitivity adjusted model, a statistical model, a mathematical model, and a machine learning technique.
24A. The method of innovation 14A wherein said using a model is executed remote from an open platform having temperature and fluid accumulation sensors for detecting the first fluid accumulation information and the earlier foot temperature information.
25A. The method of innovation 14A further comprising receiving answers from the patient from a plurality of questions, the model using at least one of the answers and items 1-4 to determine whether a pattern indicative of heart failure decompensation exists.

What is claimed is:

1. A heart failure management system comprising:
an input configured to receive multi-modal physiological information from a platform, the platform having at least one temperature sensor configured to produce foot temperature information associated with the temperatures of at least one portion of a patient's foot, the platform further having at least one weight sensor configured to produce patient weight information, the platform including at least one of an open platform and a closed platform;
an analysis engine with a multi-modal model configured to analyze physiological information received at the input, the analysis engine configured to receive first foot temperature information produced by the at least one temperature sensor, the model also configured to receive earlier foot temperature information of the patient, the first foot temperature information being temporally spaced from the earlier foot temperature information, the first foot temperature information and earlier foot temperature information of the patient each associated with at least one portion of the patient's foot,
the analysis engine further configured to receive first weight information and earlier weight information of patient, the first weight information being temporally spaced from the earlier weight information, the multi-modal model of the analysis engine configured to determine whether 1) the first weight information, 2) the earlier weight information, 3) the first foot temperature information, and 4) the earlier foot temperature information collectively shows a pattern indicative of heart failure decompensation; and
an output configured to produce output information for use in clinical decision-making or patient monitoring relating to whether the multi-modal model determined a pattern indicative of heart failure decompensation.

2. The system of claim 1 further comprising the platform.

3. The system of claim 2 wherein the platform comprises an open platform.

4. The system of claim 3 wherein at least a portion of the analysis engine is remote from the open platform.

5. The system of claim 1 wherein the analysis engine is configured to:
determine whether the first weight information and earlier weight information indicate a change of weight; and
determine whether the first foot temperature information and the earlier foot temperature information indicate a change in foot temperature,
the model using the determinations of both change of weight and change of foot temperature to determine whether 1-4 collectively indicates heart failure decompensation.

6. The system of claim 1 wherein the first foot temperature information are associated with a given two or more portions of the patient's foot, the earlier foot temperature information also are associated with the given two or more portions of the patient's foot.

7. The system of claim 1 further comprising a display to display output indicia relating to whether output information includes information relating to a pattern indicative of heart failure decompensation.

8. The system of claim 1 wherein the earlier foot temperature information comprises a representation of the temperature of the whole foot as a median or average temperature across measurements before the earlier temperature measurement.

9. The system of claim 1 wherein the model is configured to use fluid accumulation to determine whether a pattern indicative of heart failure decompensation exists.

10. The system of claim 7 further comprising at least one bioimpedance sensor to determine fluid accumulation in the patient.

11. The system of claim 1 wherein the model may include one or more of a sensitivity adjusted model, a statistical model, a mathematical model, and a machine learning technique.

12. The system of claim 1 further wherein the model is configured to receive answers from the patient from a plurality of questions, the model using at least one of the answers and items 1-4 to determine whether a pattern indicative of heart failure decompensation exists.

13. A method of identifying a risk of heart failure decompensation of a patient, the method comprising:
receiving first weight information and earlier weight information of the patient, the first weight information being temporally spaced from the earlier weight information;
receiving first foot temperature information and earlier foot temperature information of a foot of the patient, the first foot temperature information and earlier foot temperature information of the patient each associated with at least one portion of the patient's foot, the first foot temperature information being temporally spaced from the earlier foot temperature information,
at least one of the first weight information and the first foot temperature information being produced by and communicated from a platform having at least one weight sensor to detect weight and at least one temperature sensor configured to determine the temperature at different spaced apart portions of the foot, the platform including one of an open platform and a closed platform;
using a multi-modal model executing on at least one computing device and configured to analyze physiological data to determine whether 1) the first weight information, 2) the earlier weight information, 3) the first foot temperature information, and 4) the earlier foot temperature information collectively shows a physiological pattern indicative of heart failure decompensation; and
producing output information for use in clinical decision-making or patient monitoring relating to whether the model determined a pattern indicative of heart failure decompensation.

14. The method of claim 13 wherein using comprises:
determining whether the first weight information and earlier weight information indicate a change of weight; and
determining whether the first foot temperature information and the earlier foot temperature information indicate a change in foot temperature,
the model using the determinations of both change of weight and change of foot temperature to determine whether 1-4 collectively indicates heart failure decompensation.

15. The method of claim 13 wherein the first foot temperature information are associated with a given two or more portions of the patient's foot, the earlier foot temperature information also are associated with the given two or more portions of the patient's foot.

16. The method of claim 13 further comprising displaying on a display device output indicia relating to whether the model determined a pattern indicative of heart failure decompensation.

17. The method of claim 13 wherein the earlier weight information comprises a baseline of weight information, further wherein the earlier foot temperature information comprises a baseline of temperature information.

18. The method of claim 13 wherein the earlier foot temperature information comprises a representation of the temperature of the whole foot as a median or average temperature across measurements before the earlier temperature measurement.

19. The method of claim 13 further comprising receiving fluid accumulation information relating to the patient, said using a model comprising also using fluid accumulation information to determine whether a pattern indicative of heart failure decompensation exists.

20. The method of claim 19 further comprising using bioimpedance sensors to determine fluid accumulation in the patient.

21. The method of claim 13 wherein receiving first weight information comprises receiving the weight information from an open platform having weight sensors.

22. The method of claim 13 wherein receiving foot temperature information comprises receiving the foot temperature information from an open platform having one or more temperature sensors.

23. The method of claim 13 wherein the model may include one or more of a sensitivity adjusted model, a statistical model, a mathematical model, and a machine learning technique.

24. The method of claim 13 wherein said using a model is executed remote from an open platform having temperature and weight sensors for detecting the first weight information and the earlier foot temperature information.

25. The method of claim 13 further comprising receiving answers from the patient from a plurality of questions, the model using at least one of the answers and items 1-4 to determine whether a pattern indicative of heart failure decompensation exists.

26. The method of claim 13 further comprising displaying indicia on the platform during use via a user display.

27. A computer program product for use on a computer system for identifying a risk of heart failure decompensation of a patient, the computer program product comprising a tangible, non-transient computer usable medium having computer readable program code thereon, the computer readable program code comprising:
program code for receiving first weight information and earlier weight information of the patient, the first weight information being temporally spaced from the earlier weight information;
program code for receiving first foot temperature information and earlier foot temperature information of patient, the first foot temperature information and earlier foot temperature information of the patient each associated with at least one portion of the patient's foot, the first foot temperature information being temporally spaced from the earlier foot temperature information,
at least one of the first weight information and the first foot temperature information being produced by and communicated from a platform having at least one weight sensor to detect weight and at least one temperature sensor configured to determine the temperature at different spaced apart portions of the foot, the platform including one of an open platform and a closed platform;
program code for using a multi-modal model executing on at least one computing device and configured to analyze physiological data to determine whether 1) the first weight information, 2) the earlier weight information, 3) the first foot temperature information, and 4) the earlier foot temperature information collectively shows a physiological pattern indicative of heart failure decompensation; and
program code for producing output information for use in clinical decision-making or patient monitoring relating to whether the model determined a pattern indicative of heart failure decompensation.

28. The computer program product of claim 27 wherein the program code for using comprises:
program code for determining whether the first weight information and earlier weight information indicate a change of weight; and
program code for determining whether the first foot temperature information and the earlier foot temperature information indicate a change in foot temperature,
the model using the determinations of both change of weight and change of foot temperature to determine whether 1-4 collectively indicates heart failure decompensation.

29. The computer program product of claim 27 wherein the first foot temperature information are associated with a given two or more portions of the patient's foot, the earlier foot temperature information also are associated with the given two or more portions of the patient's foot.

30. The computer program product of claim 27 further comprising program code for displaying on a display device output indicia relating to whether the model determined a pattern indicative of heart failure decompensation.

31. The computer program product of claim 27 wherein the earlier weight information comprises a baseline of weight information, further wherein the earlier foot temperature information comprises a baseline of temperature information.

32. The computer program product of claim 27 wherein the earlier foot temperature information comprises a representation of the temperature of the whole foot as a median or average temperature across measurements before the earlier temperature measurement.

33. The computer program product of claim 27 further comprising program code for receiving fluid accumulation information relating to the patient, said program code for using a model comprising program code for also using fluid accumulation to determine whether a pattern indicative of heart failure decompensation exists.

34. The computer program product of claim 27 wherein the program code for receiving first weight information comprises program code for receiving the weight information from an open platform having weight sensors.

35. The computer program product of claim 27 wherein the model may include one or more of a sensitivity adjusted model, a statistical model, a mathematical model, and a machine learning technique.

36. The computer program product of claim 27 wherein the program code for using a model is executed remote from an open platform having temperature and weight sensors for detecting the first weight information and the earlier foot temperature information.

37. The computer program product of claim 27 further comprising program code for receiving answers from the patient from a plurality of questions, the model using at least one of the answers and items 1-4 to determine whether a pattern indicative of heart failure decompensation exists.
